(19) United States
(12) Patent Application Publication
(10) Pub. No.: US 2025/0228519 A1
(43) Pub. Date: Jul. 17, 2025

(54) PATIENT INTERFACE SYSTEM

(71) Applicant: Delphinus Medical Technologies, Inc., Novi, MI (US)

(72) Inventors: Carri K. GLIDE-HURST, Novi, MI (US); Nebojsa DURIC, Novi, MI (US); Peter J. LITTRUP, Novi, MI (US)

(73) Assignee: Delphinus Medical Technologies, Inc., Novi, MI (US)

(21) Appl. No.: 18/930,910

(22) Filed: Oct. 29, 2024

Related U.S. Application Data
(63) Continuation of application No. 16/212,760, filed on Dec. 7, 2018, now Pat. No. 12,161,505.
Application No. 16/212,760 is a continuation of application No. 14/486,551, filed on Sep. 15, 2014, now Pat. No. 10,201,324.
Application No. 14/486,551 is a continuation-in-part of application No. 12/115,174, filed on May 5, 2008, now Pat. No. 8,870,771.
(60) Provisional application No. 60/915,946, filed on May 4, 2007.

(51) Int. Cl. A61B 8/08; A61B 5/00; A61B 8/00; A61B 8/15

(52) U.S. Cl. CPC A61B 8/0825; A61B 8/085; A61B 8/0858; A61B 8/15; A61B 8/406; A61B 8/4494; A61B 8/5223; A61B 5/4312; A61B 8/4281

(57) ABSTRACT

A method for categorizing whole-breast density is disclosed. The method includes the steps of exposing breast tissue to an acoustic signal; measuring a distribution of an acoustic parameter by analyzing the acoustic signal; and obtaining a measure of whole-breast density from said measuring step. An apparatus is also disclosed.

### CROSS-REFERENCE TO RELATED APPLICATIONS

This application is a continuation application of U.S. application Ser. No. 16/212,760 filed 7 Dec. 2018, which is a continuation application of U.S. application Ser. No. 14/486,551 filed 15 Sep. 2014, now U.S. Pat. No. 10,201,324, issued 12 Feb. 2019, which is a continuation-in-part application of U.S. application Ser. No. 12/115,174 filed 5 May 2008, now U.S. Pat. No. 8,870,771, issued 28 Oct. 2014, which claims the benefit of U.S. Provisional Application Ser. No. 60/915,946 filed 4 May 2007, which are each incorporated in their entirety herein by this reference.

### STATEMENT AS TO FEDERALLY SPONSORED RESEARCH

This invention was made with Government support under Grant R44CA165320 awarded by the National Institutes of Health (NIH) through the National Cancer Institute. The Government has certain rights to this invention.

### TECHNICAL FIELD

This disclosure relates to medical devices and more particularly relates to a method and apparatus for determining breast density and assessing cancer risk utilizing acoustic parameters.

### BACKGROUND

According to the World Health Organization, breast cancer is the second most common type of cancer and the fifth most common cause of cancer death. In view of the commonality of breast cancer, diligent individuals subject themselves to regular mammograms for the purpose of detecting an existence of breast cancer.

An ancillary benefit of having a mammogram conducted is the ability of the radiologist to determine a radiographical density of the participant's breast tissue due to the fact that there is a prognostic relationship between breast density and cancer risk. In general, it is known in the art that the radiographical density of a breast illustrated within a mammogram may vary due to differences in the amount of fat, connective tissues, and epithelial tissues that are present. For example, because fibroglandular and connective tissues (i.e. glands, ducts, and fibers) have a relatively high x-ray attenuation, fibroglandular and connective tissues may appear to be radiographically dense/light on radiographic films. By contrast, fat has a relatively low x-ray attenuation and therefore appears to be the least radiographically dense/dark, when compared to the remaining breast tissue. Because of the distinct differences in x-ray attenuation between fat and fibroglandular tissue, segmentation of fibroglandular tissue from the rest of the breast is possible.

A known breast density estimation standard may be based upon a four-category Breast Imaging Reporting and Data Systems (BI-RADS) lexicon. Upon visually assessing a mammogram, a radiologist may classify the radiographical image of the breast into one of four BI-RADS compositional categories defined as: 1: Fatty, 2: Scattered, 3: Heterogeneous, and 4: Dense. Women whose breasts are categorized in the BI-RADS 4/densest breast category are four-to-six times more likely to develop breast cancer than those categorized as BI-RADS 1/fatty

Because the above standard in breast density estimation involves a radiologist's visual assessment of a mammogram, this assessment is subjective and relies on the perception of the radiologist. While such a subjective density classification is quick to use and widely employed, it has been proven to be limited due to considerable intra- and inter-reader variability of a radiologist.

Further, as also known in the art, the use of mammography is not ideal because of the associated radiation exposure to the participant that is undergoing breast evaluation Even further, a mammogram is a two-dimensional projection which, by definition, does not provide an accurate, three-dimensional volumetric estimation of the breast density due to the tissue thickness not being taken into account. Even further, it is also known that women are apprehensive about mammography due to the uncomfortable compression of the breast associated with the scan.

Accordingly, there is a need in the art for an improved method and apparatus for determining breast density. In an embodiment, the novel method and apparatus is a non-ionizing method and apparatus. In an embodiment, the determined breast density may be utilized for assessing cancer risk.

Further, because the novel methodology yields parameters that are associated with high breast density, which, in turn, may be associated with increased breast cancer risk, it will be appreciated that data arising from the novel methodology may be utilized to assess cancer risk directly without the intermediate step of determining breast density.

Yet even further, by identifying women having high breast density, further preventative measures can be implemented.

### BRIEF DESCRIPTION OF THE FIGURES

FIG. 1 is an environmental view of a non-ionizing, non-compressing, volumetric ultrasound tomography apparatus in accordance with an exemplary embodiment of the invention;

FIG. 2 is a perspective, cross-sectional view of an ultrasound transducer ring of the ultrasound tomography apparatus of FIG. 1 relative a breast in accordance with an exemplary embodiment of the invention;

FIG. 3 is a tissue sound speed frequency histogram of a subject obtained from the apparatus of FIG. 1;

FIG. 4 is a plot of mean tissue sound speed for a group of subjects according to a BI-RADS compositional categorization;

FIG. 5 is a block diagram of an apparatus for determining ultrasound percent density in accordance with an exemplary embodiment of the invention;

FIG. 6A is a plot of tissue density factor (TDF) according to quantitatively obtained mammographic percent density;

FIG. 6B is a plot of tissue density factor (TDF) according to quantitatively obtained mammographic percent density;

FIGS. 7A-7D each depict a graphical representation of intensity projections of three-dimensional sound speed data for a typical breast from each BI-RADS compositional category (1: fatty to 4: dense), wherein the white regions demonstrate high sound speed values relative to the darker lower sound speed regions; and

FIGS. 8A-8H each illustrates a graphical representation of a lesion associated with breast tissue over time based upon acoustic parameters.

### DESCRIPTION OF THE PREFERRED EMBODIMENTS

The Figures illustrate an exemplary embodiment of a method and apparatus for determining breast density and assessing cancer risk utilizing acoustic parameters in accordance with an embodiment of the invention. Based on the foregoing, it is to be generally understood that the nomenclature used herein is simply for convenience and the terms used to describe the invention should be given the broadest meaning by one of ordinary skill in the art. Further, although an embodiment of the invention described in the foregoing disclosure is related to the analysis of breast tissue, it will be appreciated that the invention is not meant to be limited to breast tissue; for example, it will be appreciated that the invention may be utilized to analyze organ tissue, such as, for example, a liver. As such, the foregoing methodologies disclosed in the foregoing disclosure may be utilized to assess the risk of, for example, cancer of the liver.

### 1. Apparatus

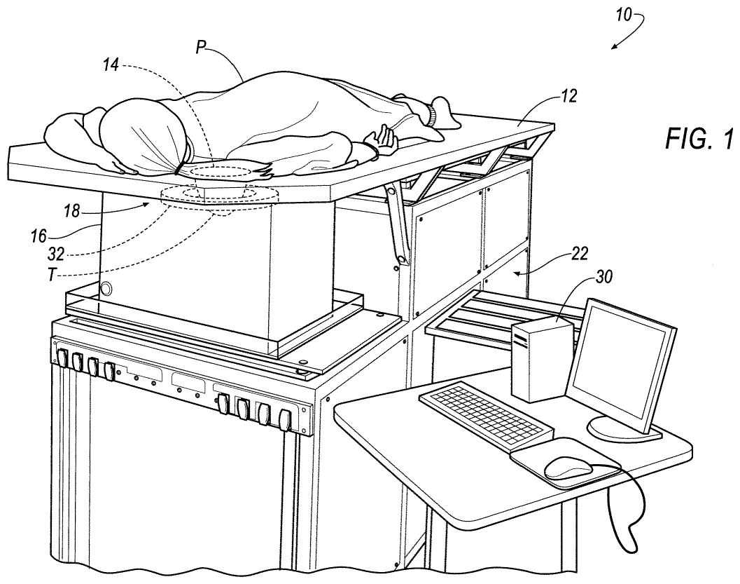

Referring to FIG. 1, an environmental view of a non-ionizing, non-compressing, volumetric ultrasound tomography apparatus 10 is shown according to an embodiment. According to an embodiment, the apparatus 10 includes a platform 12 having an opening 14 and a tank 16 having an imaging device 18 disposed in the tank 16.

Because the apparatus 10 utilizes ultrasound to analyze the tissue, T, there is no ionizing radiation exposure to the patient, P. In addition, because the tissue is immersed in a fluid within the tank 16, there is no compression of the tissue, T, which may otherwise cause discomfort to the patient, P. Even further, it will be appreciated that the apparatus 10 provides a volumetric, three-dimensional analysis of the tissue, T, as opposed to a less accurate, two-dimensional analysis of the tissue, T. Finally, it will be appreciated that the time it takes for apparatus 10 to scan a breast tissue volume is typically less than 90 seconds.

In an embodiment, the patient, P, is a female, and the tissue, T, being analyzed is breast tissue. However, it will be appreciated that the patient, P, being examined is not limited to being a female, and, the tissue, T, being scrutinized is not limited to breast tissue.

In operation and as shown in FIG. 1, the patient, P, is situated in a face-down position on the platform 12 such that the breast tissue, T, is inserted through the opening 14 and into the tank 16. As illustrated, the opening 14 is substantially aligned with the location of the tank 16, which is located under the platform 12, relative the patient, P. Once inserted through the opening 14, the breast tissue, T, is immersed in fluid within the tank 16.

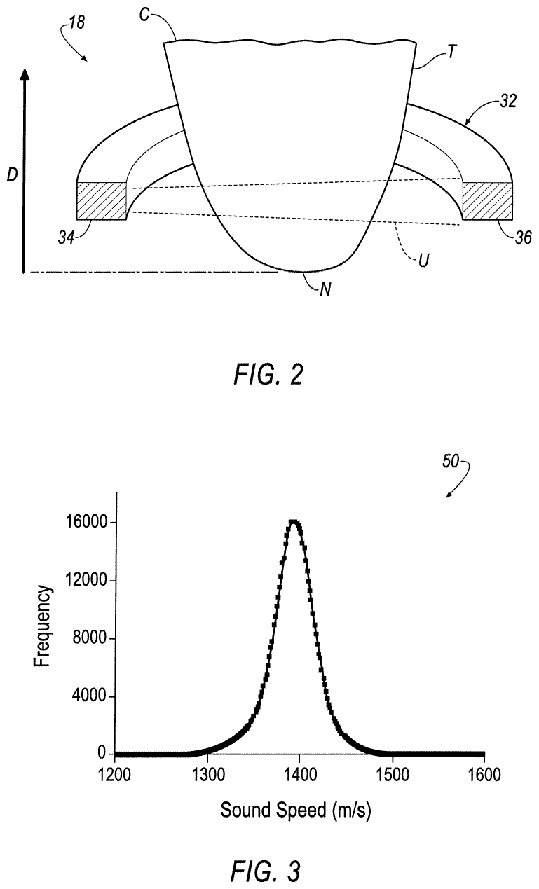

Referring to FIG. 2, the imaging device 18 is shown according to an embodiment. The imaging device 18 may be an ultrasound tomography device comprising a transducer ring 32. As shown in FIG. 1, the transducer ring 32 of the imaging device 18 is immersed in the fluid. As is shown, the transducer ring 32 includes a transmitter end 34 and a receiver end 36.

According to an embodiment, the transmitter end 34 and receiver end 36 may be constructed from any desirable material, such as, for example, an array of piezoelectric elements, an array of ceramic elements, an array of electromechanical elements, an array of magnetostrictive elements, or the like. Even further, in an embodiment, it will be appreciated that the array may be static, a stationary pair, a rotated pair, or the like. In an embodiment, the array may comprise a two-dimensional array, or, alternatively, a three-dimensional array that may be translated along different planes to enable three-dimensional data collecting, which is described in greater detail below.

In an embodiment, an ultrasound caliper device including a transmitting end and receiving end, can also be used in a substantially similar manner as the transducer ring 32 in order to collect data. If an ultrasound caliper is utilized, acoustic parameters can be obtained and evaluated without the construction of a graphical image. Further, the use of an ultrasound caliper provides a simple, hand-held device and therefore eliminates the use of a relatively larger tank 16 including, for example, a water bath.

Referring to FIGS. 1 and 2, once the breast tissue, T, is inserted into the tank 16, the breast tissue, T, is circumscribed by the transducer ring 32. Thus, ultrasound analysis of the breast tissue, T, may be conducted by transmitting sound waves, U, from the transmitter end 34 to the receiver end 36 by way of the fluid and breast tissue, T, disposed within the tank 16.

To obtain a volumetric, three-dimensional analysis of the breast tissue, T, the transducer ring 32 is initialized to a start position within the tank 16. Once initialized to the desired starting position, the sound-waves, U, are emitted as the transducer ring 32 moves in a direction according to the arrow, D, toward the platform 12/chest, C, of the patient, P, to an end position. The fluid, which may be, for example, water, is known to have well-defined sound speed parameters that serve as both a coupling medium and a matching layer between the breast tissue, T, and the transducer elements 34, 36.

Although the above-disclosed embodiment is described to include an apparatus 10 having a tank 16 holding a fluid, it will be appreciated that the invention is not limited to the use of the apparatus 10 for obtaining tissue data of a breast. For example, coupling fluids other than water, such as, for example, acoustic gels, may be applied to an outer surface of the breast; and, in an embodiment, a transducer may directly contact the breast by way of the acoustic gel. In another embodiment, a single transducer in conjunction with acoustic mirrors may also be utilized instead of the apparatus 10. A further alternative embodiment may include measurements in the time domain such that collected data from one or more transducers is not related to imaging data associated with the apparatus 10.

### 2. Determining Breast Density

In an embodiment, the sound waves, U, may be utilized to determine whole-breast acoustic measures. In an embodiment, the sounds waves, U, are utilized to determine an average whole breast acoustic sound speed velocity. The average whole breast acoustic sound speed velocity may then be utilized as an indicator of the density of the breast tissue, T.

In an embodiment, the speed of the sound waves, U, is based on signals transmitted through the breast tissue, T, and are used to generate graphical maps of the sound speed distribution. In an embodiment, the sound speed may be provided in a scale of, for example, meters-per-second (m/s). In another embodiment, graphical maps of sound speed distribution are not created such that a sound speed measurement, calculation or the like representing an entire breast may be provided. In an embodiment, the sound speed measurement, calculation or the like may be utilized as a factor for assessing cancer risk.

In an embodiment, whole breast acoustic velocity may be defined as a global sound speed measure obtained from a sound speed histogram 50 (see, e.g., FIG. 3), which is developed from tomograms that are produced, in an embodiment, by the apparatus 10. Referring to Equation 1 below, under the assumption that the tissue, T, is fluid-like, the speed of sound (V) has the following relationship to an elastic constant (c) and material density (p) of the material through which the sound waves, U, travel:

V=sqrt(c/p) (1)

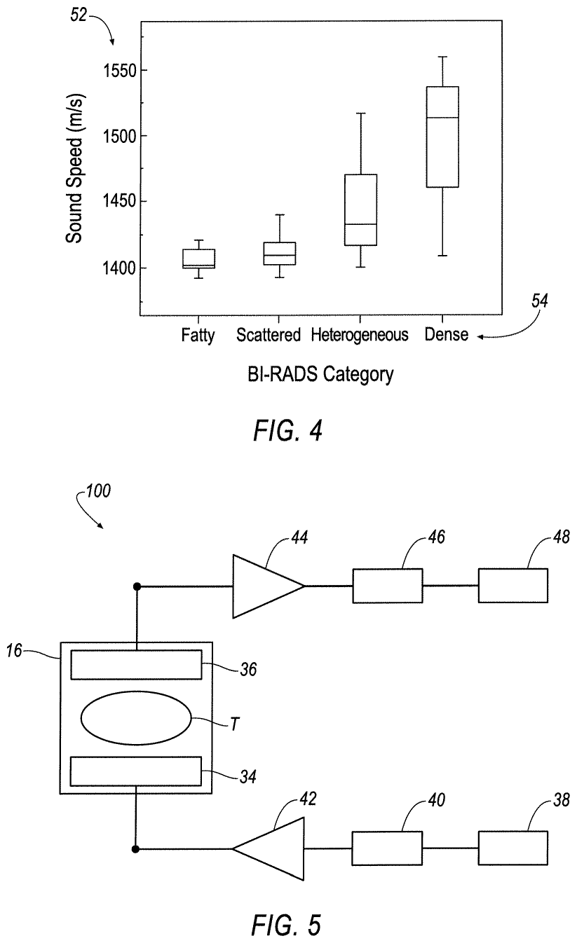

From this relationship, the average velocity 52 (see, e.g. FIG. 4) through tissue, T, would be related to the breast density and elasticity. Water, which has similar properties to tissue, T, has a bulk elastic constant of 2.2.times. 10.sup.9 N/m.sup.2. In vitro and in vivo data both support the relationship of increased sound speed with increased density of material.

To provide the histograms 50, image stacks are created for each segment of the patient's breast as the transducer ring 32 moved from its initialized start position to its end position. Because an image stack corresponds to the entire breast volume, the histograms 50 represent the statistical distribution of all sound speed voxels within that particular breast. From the histogram 50, an overall mean sound speed value for each breast is obtained to provide a single-value estimate of the volumetric average of the sound speed velocity 52 of the whole breast.

It will be appreciated, however, that measurements of an overall mean is not intended to limit the present invention and that other measures may be utilized. For example, other measures may include, but are not limited to a median, mode, midrange, skewness, kurtosis, or the like.

It will be appreciated that an overall sound speed is not meant to limit the present invention and that other measures of acoustical data may be utilized. For example, other acoustical data may include, but are not limited to attenuation, reflection, elasticity, or the like. Also, derivations of acoustical properties other than measures of central tendency may be used to derive a whole breast density measure. Examples could include parameters such as texture (2D or 3D, skewness, kurtosis, etc.) and pattern (fractal dimension, lacunarity, etc.). Even further, data integration could also be used for whole breast density acoustical measures, such as combining features from several temporal scans, different acoustical parameters, etc.

Referring to FIG. 4, as a basis to correlate breast density and the average acoustic sound speed velocity 52, a sampling of average sound speed histograms 50 were arranged against corresponding qualitative BI-RADS breast density measures 54 for each subject of the sampled population. As is known in the art, BI-RADS compositional categories are a known breast density estimation standard (i.e., classification in BI-RADS: 1-3 means there is a lesser cancer risk than a classification of BI-RADS: 4). The use of BI-RADS in FIG. 4 is for comparative purposes to illustrate the inventive concept of utilizing non-ionizing acoustics to quantify a cancer risk assessment of a biological tissue such as, for example, breast tissue, T.

Accordingly, to establish the data shown in FIG. 4, each patient, P, in the sampled population were subjected to an acoustic signal such that whole-breast sound speed data could be measured and analyzed for obtaining an acoustic measure of each patient's breast tissue, T. Prior to or after obtaining the acoustic measure, a BI-RADS classification for each patient was also established. Accordingly, as illustrated in FIG. 4, data for the entire sampled population was compiled. It can be seen that as the average acoustic sound speed velocity 52 of each patient's breast tissue, T, increases, there is also an increase, in a corresponding fashion, with a radiologist's qualitative assessment of the BI-RADS density of each subject patient, P.

In an embodiment, the relationships of the data shown in FIG. 4 are expressed as follows. According to an embodiment, average volumetric sound speed velocities 52 ranging between approximately 1400-1415 m/s relate to a fatty (BI-RADS: 1) categorization. According to an embodiment, average volumetric sound speed velocities 52 ranging between approximately 1405-1420 m/s relate to a scattered (BI-RADS: 2) categorization. According to an embodiment, average volumetric sound speed velocities 52 ranging between approximately 1420-1465 m/s relate to a heterogeneous (BI-RADS: 3) categorization. According to an embodiment, average volumetric sound speed velocities 52 ranging between approximately 1460-1575 m/s relate to a dense (BI-RADS: 4) categorization.

According to an alternative embodiment, average volumetric sound speed velocities 52 ranging between approximately 1438-1450 m/s relate to a fatty (BI-RADS: 1) categorization. According to an embodiment, average volumetric sound speed velocities 52 ranging between approximately 1450-1463 m/s relate to a scattered (BI-RADS: 2) categorization. According to an embodiment, average volumetric sound speed velocities 52 ranging between approximately 1463-1476 m/s relate to a heterogeneous (BI-RADS: 3) categorization. According to an embodiment, average volumetric sound speed velocities 52 greater than 1476 m/s (e.g., ranging between approximately 1476-1489 m/s) relate to a dense (BI-RADS: 4) categorization.

It will be appreciated that by using the above-described data associated with FIG. 4 or data according to the alternative embodiment as a frame of reference, sound speed threshold values can be determined and utilized alone (without determining density of breast tissue, T) to identify women who are at increased breast cancer risk. To serve as an example, if a patient's breast tissue, T, is defined to have a sound speed measure of, for example, 1402 m/s (or 1442 m/s), the measure falls into the range of 1400-1415 m/s (or 1438-1450 m/s), and, as such, the patient, P, may be said to have a lesser risk of developing breast cancer in the future due to the fact that the 1402 m/s (or 1442 m/s) measure falls in the range of 1400-1415 m/s (or 1438-1450 m/s), which is associated with a fatty/BI-RADS: 1 categorization.

Thus, the four sound speed ranges described above may establish, in an embodiment, the basis of a cancer risk model such that a sound speed measure falling into the range of 1460-1575 m/s (or 1476-1489 m/s) may be said to have a greatest risk of developing breast cancer than sound speed measures other ranges. Accordingly, by obtaining a measure (e.g., sound speed) and comparing the measured value against the above ranges, a quantified risk assessment may be obtained.

It can be appreciated that the basis of a breast cancer risk evaluation model using acoustic parameters is not limited to comparisons with BI-RADS compositional categories, and instead, more quantitative evaluation methods can be considered (i.e. computer-assisted segmentation of mammograms using interactive thresholding or automated segmentation). Further, it can be appreciated that a breast cancer risk evaluation model can be developed independent of any association with other breast density evaluation techniques.

It will be appreciated, however, that although sound speed is described above, a measured distribution of an acoustic signal is not limited to sound speed; for example, the measured distribution of acoustic signals may also be applied to attenuation, reflectivity, elasticity and the like. Further, the cancer risk model may be further refined by also applying additional data to, for example, the measured sound speed. In an embodiment, the additional data may include, for example, one or more of age, menopausal status, age at menarche, parity, age at first birth, number of first and second degree relatives with breast cancer, prior breast biopsies, hormonal usage, history of atypical hyperplasia, or history of lobular carcinoma in situ, and the like.

### 3. Determining Breast Density Using a Tissue Volume Separator

Referring now to FIG. 5, a block diagram 100 for a tissue volume separator from acoustic data provided by the apparatus 10 is shown generally at 100 according to an embodiment. The block diagram generally includes a waveform generator 38, a digital-to-analog converter 40, an acoustic transmit amplifier 42, the transmitter end 34, the receiver end 36, an acoustic receive amplifier 44, an analog-to-digital converter 46, and tissue volume separator value generator 48.

Functionally, the waveform generator 38 creates a series of digital signals. The digital signals are sequentially sent to the digital-to-analog converter 40 that converts each signal to a corresponding analog out-put signal which is then amplified by the transmit amplifier 42. The amplified signal drives the transmitter end 34, which converts the electrical analog signal into an acoustical signal, which propagates through the fluid in the tank 16 to the receiver end 36. The receiver end 36 converts the acoustical signal back into an electrical analog received signal, which may be amplified by a receive amplifier 44. The received signal may be digitized by the analog-to-digital converter 46.

The sampling rate of the analog-to-digital converter 46 can be set to digitize the analog signal at the Nyquist (or other) rate corresponding to the frequency used to drive the transducers 34, 36. The resulting digital data stream may be stored in the tissue volume separator 48, which may include a digital storage system in the form a random access memory, a hard drive, or the like. It will be appreciated that the tissue volume separator may be a stand-alone device, or, alternatively, be incorporated in the electronics 22, or, alternatively be incorporated in the central processing unit 30.

To determine the tissue volume separator results of the breast tissue, T, the processor of the breast density factor 48 is concerned with breast sound speed. According to an embodiment as seen in Equation 2 below, a tissue density factor, TDF, can be defined by:

TDF=(High_Sound_Speed_Volume)/(Total_Breast_Volume) (2)

The denominator (i.e., Total Breast Volume) is further defined as the total (summed) integrated areas of breast sound speed tomograms. The numerator (i.e., High Sound Speed Volume Data) is further defined as the integrated area of sound speed regions from the sound speed tomograms that are considered to be of high sound speed value. The can be determined by the areas exceeding a sound speed threshold value. This threshold for determining high sound speed is not meant to limit the present invention and that other techniques used to segment higher sound speed regions may be utilized. For example, other techniques may include, but are not limited to semi-automated or automated: k-means or fuzzy clustering, segmentation in the frequency domain or time domain, artificial neural networks, or the like. Further, the TDF can be determined from data arising directly from a three-dimensional (3D) volume as opposed to a stack of two dimensional (2D) tomograms.

It can be appreciated that using this methodology, sound speed threshold values can be determined and applied to an entire population, thereby associating TDF value of a subject to identify the likelihood of breast cancer risk. Similarly, tissues other than fatty and fibroglandular may be further segmented by volumetric assessment of other ultrasound parameters. In an embodiment, it will be appreciated that the TDF is not restricted to a ratio, but also, an absolute integrated area of dense tissue and total breast tissue as indicators of breast cancer risk.

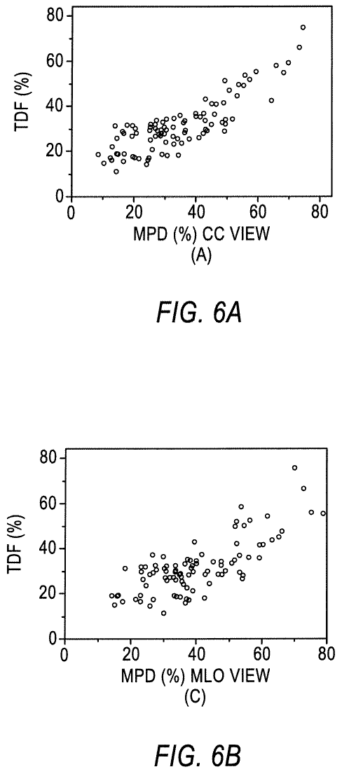

Referring to FIGS. 6A and 6B, as a basis to correlate TDF and breast density, the TDF is shown along the y-axis and quantitative mammographic percent density using semi-automated segmentation routines is shown along the x-axis. The TDF was calculated according to Equation 2 above for a plurality of patients and the results are correlated to a corresponding quantitative mammographic percent density (MPD) measures for each patient of the plurality of patients. For illustrative purposes, FIG. 6A provides an MPD for the craniocaudal (CC) view and FIG. 6B provides an MPD for the mediolateral oblique (MLO) (MLO) view. In general, FIGS. 6A and 6B provides evidence that as TDF of the breast tissue, T, increases, the MPD increases in a corresponding fashion.

It will be appreciated that, similar to providing several different volumetric distributions of imaging data, the associated tissue characterizations and assorted tissue types can be displayed in multiple different distributions. While sound speed has been currently used to separate fatty from parenchymal breast tissues for volumetric density estimates, other imaging data (such as attenuation, reflectivity, elasticity, etc.) may allow further volumetric separation of cystic areas, and benign and malignant tumors from the current separation of fatty and parenchymal tissue by sound speed alone.

### 4. Applications of Acoustic and TDF Measurements of the Breast

Multiple measurements of the breast can be conducted in space in a repeated fashion by positioning the transducer ring 32 and discretely measuring the breast tissue, T, in two-dimensional cycles until full, three-dimensional coverage of the breast tissue, T, is achieved. As such, discrete, two-dimensional measurements of the breast tissue, T, may be obtained to provide coverage of the breast tissue, T, at different cross-sections to simulate a three-dimensional analysis of the breast tissue, T, by ‘stacking’ a plurality of discrete, two-dimensional measurements. Although it is possible to ‘stack’ a plurality of discrete, two-dimensional measurements to provide a three-dimensional measurement, it will be appreciated that a direct three-dimensional measurement with a two-dimensional array is also possible.

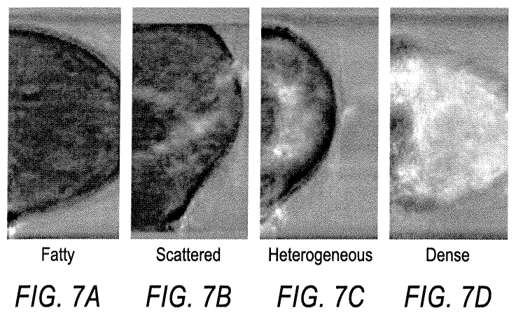

It will be appreciated that having three-dimensional reconstructions of the breast also can allow for 2D or 3D projection images of the acoustical parameters such that a direct comparison to mammography can be made. In an embodiment, as shown in FIGS. 7A-7D, maximum intensity projections of graphical sound speed maps are shown such that exemplar images of the four BI-RADS categorizes are represented. As seen in FIG. 7A, for example, a sound speed map of a BI-RADS 1/fatty tissue, T, is shown. As seen in FIG. 7B, for example, a sound speed map of a BI-RADS 2/scattered tissue, T, is shown. As seen in FIG. 7C, for example, a sound speed map of a BI-RADs 3/heterogeneous tissue, T, is shown. As seen in FIG. 7D, for example, a sound speed map of a BI-RADS 4/dense tissue, T, is shown. Here, the white regions demonstrate high sound speed values relative to the darker lower sound speed regions. It will be appreciated, however, that although maximum intensity projections of three-dimensional sound speed reconstruction is provided in each of FIGS. 7A-7D, graphical representations may be extended to different projections and acoustical parameters other than sound speed.

In an embodiment, the sound speed may be estimated either through direct measurement (e.g., with a transducer pair or set of ultrasound calipers). The direct measurement of sound speed through the tissue, T, is independent of reconstructed images such that the sound speed is determined directly from the timing of the arrival of the pulses without requiring the complexity of any imaging. In another embodiment, a series of sound speed tomographic images can be formed and used to obtain the volume averaged sound speed.

According to an embodiment, the stored data may be analyzed by a software program that is stored, in an embodiment, within the tissue volume separator 48 to determine various acoustic parameters associated with the interaction of the sound waves, U, with the breast tissue, T. The novel methodology associated with the software can be used to categorize breast tissue density and evaluate parameters such as sound speed, attenuation, reflectivity, elasticity, and TDF. It can be appreciated that this software can also be used to provide a combination of such parameters that may best distinguish women with elevated breast density, and, as such, an increased risk of breast cancer. An extension of such software may include the development of a breast cancer risk assessment model to incorporate other risk factors such as age, menopausal status, age at menarche, parity, hormonal usage, age at first birth, number of first and second degree relatives with breast cancer, prior breast biopsies, history of atypical hyperplasia, or history of lobular carcinoma in situ using any number of aforementioned acoustic parameters.

Using aforementioned measurements of the breast, a new classification scheme may be developed to characterize breast density using one or more acoustic parameters, TDF, or any combination thereof. It will be appreciated that due to the multitude of acoustic parameters available and the ability to measure them repeatedly, a more quantitative breast density classification scheme may be realized than currently employed using the mammographic BI-RADS Categories.

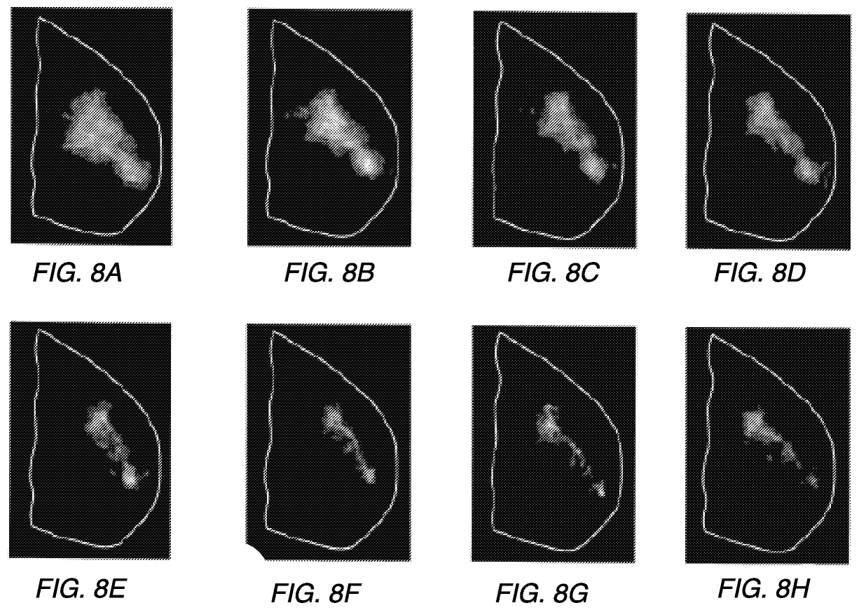

Finally, it will be appreciated that many alternative applications to volumetric tissue evaluation over time may be conducted rather than evaluating breast density over time. As noted, a diagnostic value is anticipated from volumetric tissue assessments in response to changes during the exam (e.g., temperature, shape, pressure, intravenous contrasts). Similarly, as seen in FIGS. 8A-8H, malignant tumor responses over time show volume reduction capabilities by utilizing sound speed to analyze the breast tissue, T. In an embodiment, the successive images in FIGS. 8A-8H are taken approximately two weeks apart for a patient undergoing chemotherapy. In an embodiment, changes in sound speed, tumor volume, and other acoustical properties can be tracked and estimated using a tissue volume separator. Accordingly, the invention may be utilized to analyze tumor volume reduction over time as a patient undergoes, for example, chemotherapy, radiation therapy, surgery or the like. In another embodiment, for example in benign tumors and fibrocystic breasts, the effectiveness of dietary and chemoprevention interventions maybe quantifiable in terms of density and acoustic property changes.

### 5. Benefits of Determining a Breast Density by Utilizing Acoustics

It is known that women under the age of 40 do not routinely undergo mammographic screening for breast cancer because the benefit of receiving mammographic examinations does not outweigh the risk associated with the exposure to ionizing radiation. The processes disclosed herein for determining breast density are conducted without exposing the subject to harmful ionizing radiation, and accordingly allow for risk-free screening and the establishment of baselines at an earlier age. Moreover, the present invention presents and objective approach for assessing present and future cancer risk. This is in contrast to the subjective nature of interpreting mammograms.

Even further, because the tissue undergoing examination, T, is not compressed in any of the methodologies associated with the present invention, the evaluation of the tissue, T, is conducted when the breast tissue is in a non-compressed, natural shape thereby rendering a more accurate representation of the spatial distribution of fibroglandular and fat tissues. Further, by not utilizing a compression technique, women may be more likely to respond favorably to the comfort of the scan, particularly when comparing the embodiments of the present invention to mammography.

In obtaining the breast density by way of the present methodologies, a reduced amount of time may be employed when evaluating a subject's tissue, T; the examination may take as little as five minutes to set-up and one minute to conduct. As a result of knowing one's breast tissue density, preventative measures may be taken, as the relative risk of breast cancer increases with increased breast density. Preventative measures may include, for example, the screening of such subjects at a younger age (e.g., women who are not of mammographic age, such as women that are younger than 40 years old) and/or the screening of such subjects with a greater than annual frequency. In addition, knowing one's tissue density may justify the use of chemoprevention methods. Chemoprevention methods may include, for example, dietary intervention or use of Tamoxifen.

In addition, by knowing one's acoustical or TDF measures, the known tissue density may be utilized as a baseline value for subsequent comparison over time. For example, if a subsequent screening determined breast density has increased in view of one's previous/baseline breast density, the subject may be advised to undergo earlier/preventative screenings, which may not otherwise be suggested without the knowledge of the change in breast density from the baseline.

Volumetric changes over time in response to temporal alterations of the ultrasound parameters provide further potential tissue characterization. Volumetric tissue responses can be measured during a single or multiple scans. Parameters which may be altered during a single scan session may include, but are not limited to: temperature, shape, pressure, and vascular content. Further, it will be appreciated that these more immediate volumetric changes may include all or a portion of the scanned volume. Changes in volumetric ultrasound parameters affecting vascular content may be in response to any variety of intravenous or interstitial agents which change the reflectivity, sound speed, attenuation, elasticity, etc. Volumetric parameters which may be altered during multiple scan session include, but are not limited to, interval interventions, such as: responses to chemotherapy, chemoprevention, dietary changes, radiation treatment, tumor ablations or other surgical interventions.

Even further, it will be appreciated that many alternative applications of the present invention may also be made available. For example, the breast tissue density could be monitored in response to treatments such as, for example, soy isoflavones, dietary interventions, hormone-replacement therapy, and the like. In addition, the acoustical parameters and TDF could evaluate breast density over the phases of one's menstrual cycle, which may assist in determining the ideal time span to evaluate the breast. Alternatively, the breast density could be used to determine the natural variance of breast density for each patient.

The system and/or method of the preferred embodiment and variations thereof can be embodied and/or implemented at least in part as a machine configured to receive a computer-readable medium storing computer-readable instructions. The instructions are preferably executed by computer-executable components preferably integrated with the system 100 and one or more portions of the processor 220. The computer-readable medium can be stored on any suitable computer-readable media such as RAMs, ROMs, flash memory, EEPROMs, optical devices (CD or DVD), hard drives, floppy drives, or any suitable device. The computer-executable component is preferably a general or application specific processor, but any suitable dedicated hardware or hardware/firmware combination device can alternatively or additionally execute the instructions.

As a person skilled in the art will recognize from the previous detailed description and from the figures and claims, modifications and changes can be made to the preferred embodiments of the invention without departing from the scope of this invention defined in the following claims.

1. A system for analyzing a volume of breast tissue, the system comprising:
(a) a non-ionizing apparatus comprising a transducer configured to transmit acoustic signals through the volume of breast tissue;
(b) a receiver configured to receive acoustic signals and collect a measurement of a distribution of sound speed for the volume of breast tissue; and
(c) a processor operatively connected to a non-transitory computer-readable storage medium with instructions stored thereon which when executed by the processor are configured to:
(i) obtain a first average whole breast sound speed of the breast tissue based on a first distribution of sound speed
(ii) obtain a second average whole breast sound speed of the breast tissue based on a second distribution of sound speed
(iii) characterize the breast tissue based at least in part on a comparison of the first average whole breast sound speed to second average whole breast sound speed.

2. The system of claim 1, wherein the non-ionizing apparatus comprises a platform disposed substantially adjacent to a tank including a coupling fluid.

3. The system of claim 2, wherein the platform is configured to facilitate insertion of the volume of breast tissue into the tank and proximate to the transducer.

4. The system of claim 1, wherein the non-ionizing apparatus comprises:
an imaging device;
a waveform generator;
a digital-to-analog converter coupled to the waveform generator; and
an acoustic transmit amplifier coupled to the digital-to-analog converter, wherein the acoustic transmit amplifier is connected to a transmitter end of the imaging device.

5. The system of claim 1, wherein the receiver comprises a tissue volume separator transducer is configured to receive the acoustic signal transmitted through the breast tissue.

6. The system of claim 1, wherein the non-ionizing apparatus comprises an ultrasound caliper disposed upon the coupling fluid and proximate to the breast tissue.

7. The system of claim 6, wherein the ultrasound caliper comprises a transmitting end and a receiving end for transmitting the acoustic signal through the volume of breast tissue for receipt at the receiver.

8. The system of claim 4, wherein the receiver comprises:
a receiver end coupled to the imaging device;
a receive amplifier wherein the receiver amplifier coupled to the receiver end;
an analog-to-digital converter coupled to the receive amplifier; and
a value generator connected to the analog-to-digital converter.

9. The system of claim 4, wherein the imaging device is an ultrasound tomography device comprises a transducer ring configured to scan the volume of breast tissue.

10. The system of claim 1, wherein the non-transitory computer-readable storage medium further comprises instructions stored thereon which when executed by the processor are configured to,
analyze one or more risk factors in combination with the first average whole breast sound speed and second average whole breast sound speed to further refine the tissue characterization, wherein the one or more risk factors comprise: an age, menopausal status, age at menarche, parity, age at first birth, number of first and second degree relatives with breast cancer, prior breast biopsies, hormonal usage, history of atypical hyperplasia, and history of lobular carcinoma.

11. The system of claim 1, wherein the non-transitory computer-readable storage medium further comprises instructions stored thereon which when executed by the processor are configured to
(iv) generate a first tissue density factor based on a first high sound speed volume and a total breast volume and a second tissue density factor base on a second high sound speed volume and the total breast volume, and
(v) analyze the first TDF and the second TDF in combination with the first average whole breast sound speed and second average whole breast sound speed for characterizing the breast tissue.

12. The system of claim 11, wherein characterizing the breast tissue includes classifying the volume of breast tissue as a Breast Imaging Reporting and Data System (BI-RADS) category 4, a BI-RADS category 3, a BI-RADS category 2, or a BI-RADS category 1.

13. The system of claim 1, wherein obtaining at least one of the first average whole breast sound speed or the second average whole breast sound speed comprises obtaining a statistical measure of central tendency including one of a mean, a mode, a median, a range, and a standard deviation of at least one of sound speed and attenuation.

14. The system of claim 13, wherein the statistical measure of central tendency is obtained from analyzing data derived from an interaction between the acoustic signal and the volume of breast tissue.

15. The system of claim 1, wherein the non-transitory computer-readable storage medium further comprises instructions stored thereon which when executed by the processor are configured to obtain the first average whole breast sound speed at a first time points and obtain the second average whole breast sound speed at a second time point.

16. The system of claim 11, wherein at least one of the first TDF or the second TDF is compared to at least one threshold condition associated with breast density of the volume of breast based at least in part on a threshold value of average whole breast sound speed.

17. The system of claim 1, wherein extracting at least one of the first average whole breast sound speed or the second average whole breast sound speed further comprises, scanning a set of planes spanning the volume breast tissue, developing at least one average sound speed histogram characterizing a statistical distribution of all sound speed voxels for the volume of breast tissue, and extracting the at least one of at least one of the first average whole breast sound speed or the second average whole breast sound speed based on the at least one average sound speed histogram.

18. The system of claim 1, wherein the acoustic signal comprises at least one of a sequential series of waves and a sequential series of pulses.

19. The system of claim 1, wherein the non-transitory computer-readable storage medium further comprises instructions stored thereon which when executed by the processor are configured to monitor variations in first TDF at a first set of time points and the second TDF at a second set of time points.

20. (canceled)
